(19) United States
(12) Patent Application Publication
(10) Pub. No.: US 2023/0146817 A1
(43) Pub. Date: May 11, 2023

(54) OPTIMISED THERMOCHROMATIC MATERIALS

(71) Applicant: THERMETRIX LTD, Mid Glamorgan (GB)

(72) Inventors: Benjamin Paul KLUWE, South Glamorgan (GB); Russell John PAYNE, Gwent (GB); Peter PLASSMAN, South Glamorgan (GB)

(73) Assignee: THERMETRIX LTD, Mid Glamorgan (GB)

(21) Appl. No.: 17/906,428

(22) PCT Filed: Mar. 15, 2021
(86) PCT No.: PCT/EP2021/056569

§ 371 Date: Sep. 15, 2022

(30) Foreign Application Priority Data
Mar. 16, 2020 (GB) 2003754.5

(51) Int. Cl. A61B 5/01; A61B 5/00; G01K 11/165; G01K 11/18

(52) U.S. Cl. CPC A61B 5/01; A61B 5/6825; A61B 5/6829; G01K 11/18; G01K 11/165

(57) ABSTRACT

This invention relates to optimisation of the temperature range of thermochromic liquid crystal materials and to related methods and devices for temperature monitoring and measurement. The invention also relates to methods and devices for the improved registering of objects in contact with thermochromic liquid crystal materials.

### CROSS-REFERENCE TO RELATED APPLICATION

This application claims the benefit of priority of PCT application number PCT/EP2021/056569, filed Mar. 15, 2021, the contents of which are herein incorporated by reference.

### TECHNICAL FIELD

This invention relates to optimisation of the usable temperature range of thermochromic liquid crystal materials and to related methods and devices for temperature monitoring and measurement. The invention also relates to methods and devices for the improved registering of objects in contact with thermochromic liquid crystal materials.

### BACKGROUND

As the name suggests, thermochromic liquid crystals (TLCs) are able to change colour in response to temperature change. The colour change results from changes in crystal organisation and form / state (anisotropic, nematic, smectic, chiral/cholesteric, isotropic) at different temperatures. Crystal organisation and state will differ according to temperature which in turn will affect the wavelengths of light that can be absorbed and reflected by the crystals. Since different temperatures give a corresponding change in colour, TLCs are often found in thermometers for medical or other use. TLCs are also found in novelty items such as mood rings, colour changing mugs and T-shirts.

TLCs, when used in thermometers and other devices for monitoring and measuring temperature, may be applied to a substrate. For example, thermochromic liquid crystal sheets (TLCS) comprise a layer of TLC ink, typically printed or painted onto a substrate such as plastic or glass. TLCS are sensitive to temperature and can change colour in a matter of seconds upon direct contact or near-direct contact with a heat source. At the lower end of their temperature range, TLCs are usually transparent (the smectic phase), revealing the colour of the substrate. Upon warming, the TLCs gradually lose transparency as they shift into the chiral/cholesteric phase and block out the substrate colour as they assume a colour of their own. In a frequently used formulation (see, for example, the Handbook of Liquid Crystals, Goodby etal., 2014, Wiley publications; and https://www.lcrhallcrest.com//liquid-crystal-formulation-types/), the initial colour of the TLCs is dark red, which upon further warming gradually shifts through the colours of the rainbow. At the upper end of the TLC temperature range, the ultimate violet colour will then start to become increasingly transparent (shifting into the isotropic phase), again revealing the substrate colour. Other colour change patterns also exist. As the material cools, the colours and transparencies will be observed in the reverse order. TLCS are relatively inexpensive and since the colour change is reversible the TLCS may be reused making them economically attractive.

Conventionally available TLCs are designed and manufactured to monitor temperature changes over defined ranges, for example, ranges of 5° C., such as 20-25° C.; ranges of 10° C., such as 25-35° C.; and ranges of 1° C., such as 35-36° C. TLCs with a narrow range of, say, 1° C. typically have improved temperature resolution compared to TLCs with larger ranges of say 5° C. or 10° C. When TLCs are exposed to temperatures within range, the colours move through all colours of a rainbow to violet.

Whether or not a TLCS of any given temperature range can be manufactured will depend on the constraints of the chemical properties of the TLCs.

By analysing the light reflected from the TLCs, it is possible to correlate colours to absolute temperatures. This analysis may be done by eye, by comparing the colours visible on the TLCs to a reference chart correlating colours to temperatures. The analysis may also be carried out electronically for greater accuracy and speed.

The designer of a temperature measurement system based on TLCs colour analysis faces the need to find a compromise between two conflicting design goals: on the one hand the temperature interval should be small so that colour changes are mapped to a narrow range, thus improving the temperature resolution; on the other hand, the temperature range should be wide in order to capture a broad range of temperatures.

Furthermore, commercially available TLCs are limited in the temperature ranges available, with typical ranges of 1° C., 5° C., or occasionally 10° C. or more. While it is technically possible to produce bespoke temperature ranges and intervals (an interval being the degree of accuracy to, say, the nearest 0.5° C. or 1° C.), this is expensive and therefore often commercially unviable. As a result, the TLCs may often not have the optimum range for the task in question.

Measuring a change in colour from the TLCs may also be hindered by the presence of artefacts, such as shadows and reflections, or other imperfections introduced by image-capture equipment or sources of illumination.

One of the drawbacks of using conventional TLCS is that the outline and area of objects in contact with them often cannot properly be deciphered when the TLCS is transparent, i.e. when the temperature of the object in contact with the sheet falls outside of the measurement range for the TLCS. Similarly, the outline and area of parts of a contact-making object cannot properly be deciphered when they are of the same temperature as the TLCS itself, such parts being of the same colour as the TLCS.

With conventional TLCS being opaque, it is often not possible or at least difficult to generate an accurate representation of the contact-making object and in turn to derive accurate temperature values.

A further drawback of using conventional TLCS is the difficulty in ensuring that the TLCS image (in the form of a heatmap) is overlayed or superimposed correctly onto the real ‘photo’ image of the object so that temperature values can be accurately assigned to the correct corresponding part of the subject or object. This is particularly important for measurements made in a medical context, for example, where the object in question is a hand or foot.

There is therefore a need to address the aforementioned drawbacks.

### SUMMARY OF THE INVENTION

The present invention aims to address the disadvantages of the prior art by providing in a first aspect of the invention a method for monitoring or measuring temperature using thermochromic liquid crystal (TLC) material, comprising analysis of colours revealed as the TLCs transition from transparent to colour and/or from colour to transparent, including colours imperceivable by the human eye and/or colours extending beyond the temperature range set by the TLC manufacturer.

A second aspect of the invention provides a method for monitoring or measuring hand and foot temperature in a subject, comprising (i) contacting one or both hands and/or one foot or both feet of a subject with a substrate comprising thermochromic liquid crystals (TLCs), wherein said contacting generates a heatmap on the substrate; (ii) analysing the heatmap for colours revealed as the TLCs transition from transparent to colour and/or from colour to transparent, including colours imperceivable by the human eye and/or colours extending beyond the temperature range set by the TLC manufacturer; (iii) monitoring for points of elevated temperature (“hotspots”) and/or reduced temperature (“cold spots”) on said heatmap.

A third aspect of the invention provides a thermochromic liquid crystal sheet (TLCS), comprising: (a) TLC material applied to the surface of a substrate and/or embedded in a substrate, and (b) a layer of electrochromic material.

A fourth aspect of the invention provides the use of TLCs for monitoring or deriving the temperature of an object or subject in contact therewith, comprising analysing any one or more colours revealed from the contact as the TLCs transition from transparent to colour and/or from colour to transparent, including colours imperceivable by the human eye and/or colours extending beyond the temperature range set by the TLC manufacturer.

A fifth aspect of the present invention provides a system for monitoring or measuring the temperature of a subject or object, comprising:
a) TLCs, wherein contacting of the subject or object with the TLCs generates a heatmap; and
b) optionally a camera for capturing at least one image of the heatmap, preferably wherein multiple images are captured, such as through a continuous image acquisition process; and
c) computer means for analysing colours revealed on the heatmap as the TLCs transition from transparent to colour and/or from colour to transparent, including colours imperceivable by the human eye and/or colours extending beyond the temperature range set by the TLC manufacturer, and monitoring or deriving the temperature of the object or subject by correlating the colours revealed to a temperature value.

### DETAILED DESCRIPTION

According to a first aspect of the invention, there is provided a method for monitoring or measuring temperature using thermochromic liquid crystal (TLC) material, comprising analysis of colours revealed as the TLCs transition from transparent to colour and/or from colour to transparent, including analysis of colours imperceivable by the human eye and/or colours extending beyond the temperature range set by the TLC manufacturer.

The TLC material will change colour in response to the temperature of an object or subject in contact therewith.

Analysis of the colours revealed as the TLCs transition to and/or from transparent advantageously allows the temperature range derivable from the TLCs to be extended beyond the nominal range of the TLC material. Analysis of colours revealed as the TLCs transition from transparent to colour suitably allows the lower end of the temperature range to be extended beyond the nominal lower range of the TLC material. Analysis of colours revealed as the TLCs transition from colour to transparent suitably allows the upper end of the temperature range to be extended beyond the nominal upper range of the TLC material. Analysis of colours revealed as the TLCs transition from transparent to colour and from colour to transparent suitably allows both the lower and upper ends of the temperature range to be extended beyond the nominal range of the TLC material.

For example, the temperature range may be extended beyond the lower end of the nominal range of the TLC material by analysis of the colours revealed as the TLCs transition from transparent to red (this may not be red in some TLC formulations, but may be another colour). The transition from transparent to red (or another colour) represents transition of the crystals from the smectic phase to the chiral/cholesteric phase. Analysis of the TLC colours as the crystals change from the smectic phase to the chiral/cholesteric phase would also therefore allow the temperature range to be extended beyond the lower end of the nominal range of the TLC material. The colours revealed as the TLCs transition from transparent to red, or another colour, include hues which may be represented by approximately 330° to 355° in the Hue Saturation Value (HSV) colour space, where 0° is pure red.

The temperature may also be extended beyond the upper end of the nominal range of the TLC material by analysis of the colours revealed as the TLCs transition from violet (this may be a different colour in some TLC formulations) to transparent. The transition from violet (or another colour) to transparent represents transition of the TLCs from the chiral/cholesteric phase into the isotropic phase. Analysis of the TLCs as they transition from the chiral/cholesteric phase to the isotropic phase would also therefore allow the temperature range to be extended beyond the upper end of the nominal range of the TLC material. The colours revealed as the TLCs transition from violet or another colour to transparent, include hues which may be represented by approximately 240° to 360°, preferably 290° to 355°, in the Hue Saturation Value (HSV) colour space, where 0° is pure red.

Accordingly, there is provided a method for monitoring or measuring temperature using thermochromic liquid crystal (TLC) material, comprising analysis of colours revealed as the TLCs transition from the smectic phase to the chiral/cholesteric phase and/or from the chiral/cholesteric phase to the isotropic phase.

A person skilled in the art would readily be able to determine the phase of TLCs using known techniques, such as those described in Molecular Structure and the Properties of Liquid Crystals, Gray, G. W., Academic Press, London New York (1962); and Liquid Crystals (Cambridge Monographs on Physics) Chandrasekhar, S.,Cambridge University Press (1977).

“TLC material” as defined herein refers to any material comprising TLCs and which allow TLC colour changes to be observed.

TLCs are optically active mixtures of organic chemicals that are temperature sensitive, responding to temperature changes with corresponding changes in colour. TLCs can be formulated to change through a single colour, however, in the present invention the TLC formulation is one which displays multiple colours, such as the standard Red, Green, Blue (RGB) type (https://www.lcrhallcrest.com/liquid-crystal-formulation-types/). The RGB formulation has been used in strip thermometers since the late 1970s. In the RGB formulation, the TLCs always transition from transparent to red, at the lower end of the temperature range, and from violet to transparent at the upper end of the temperature range. The TLCs may be comprised in a thermochromic liquid crystal sheet (TLCS), which may be custom made or an off-the-shelf TLCS.

Advantageously, the aforementioned analysis of the colours revealed as the TLCs transition to and from transparent allows the useful range of the TLCs to be more than doubled. In one example, the lower end of the temperature range of the TLCs may be extended by at least 5%, 10%, 15%, 20%, or 25% and/or the upper end of the temperature range of the TLCs may be extended by at least 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200% or more. Advantageously, the aforementioned analysis of the colours at least doubles the measurable temperature range of TLCs. Typically, approximately 10% of this increase may be attributed to the lower end of the temperature range (transition from transparent to colour) and approximately 90% to the upper end (transition from colour back to transparent).

The colour analysis typically comprises analysing all colours visible on the TLC material, but may exclude colours starting from the transparent to red transition to the yellow to green hues, which hues may be represented by a range from between about 0° to 90° in the HSV colour space, where 0° is pure red. Partial or total exclusion of these hues from the analysis suitably reduces the influence of spurious and unwanted artefacts, which may arise from reflections of light sources or from any measurement device.

The TLC colour transition process is not linearly correlated with temperature. A given temperature increase will result in a larger change in TLC colours at the lower design temperature end than at the upper end. As an illustrative example, both the lower end colour change from red to orange and the upper end change from orange to violet cover the same spectral wavelength interval of approximately 190 nm, however, due to the non-linear behaviour of the TLC material, the lower end (red to orange) is typically associated with less than 20% of the entire temperature range, the upper range with the remaining 80%. The colour scale is compressed at the lower end and expanded at the upper end. This means that unwanted artefacts can be significantly reduced or eliminated from the analysis by eliminating approximately the lower half of the TLC spectrum (hues in the range of from between about 0° to 90° in the HSV colour space), where unwanted image artefacts are predominantly found. This elimination sacrifices only about 20% of the design temperature range, which is more than compensated for by the approximate doubling or more of the design range by analysis of colours revealed as the TLCs transition from transparent to colour and from colour to transparent.

The quantification of hue by an angle between 0 and 360 ° is known in the art, as shown for example in FIG. 4 of Loesdau et al., 2014 (Lecture Notes in Computer Science, Vol. 8509).

The colour analysis may additionally comprise analysing only the Hue (H) component of the HSV colour space and determining temperature from only the H component, rather than by analysing all three of the H S V components.

The colour analysis may be carried out by eye by correlating the colours revealed as the TLCs transition to and from transparent to a specific temperature using standard reference tables available in the art or by using a calibration table or curve fitting formula. The colour analysis may be carried out on at least one image of the TLC material, optionally on a series of images taken over a specified period of time. Multiple images may be produced though continuous image acquisition, or a series of discrete images may be derived from a continuous video stream. Where multiple images are analysed, the multiple images may be a series of images taken during one “sitting” or over the course of a single day, week, month, year or other suitable interval. The series of images may be captured and digitally stored.

The images of the TLC material are preferably heatmaps. The specific colours revealed in the images may be correlated to specific temperatures, for example, by analysing the pixels or spatial points using a calibration table or curve fitting formula, see FIG. 6 for an example. The pixels or spatial points on the images may be analysed using a curve fitting formula to derive an extrapolated temperature value, which may be for substantially each spatial point or pixel. Such extrapolation allows temperatures to be determined beyond the nominal range of the TLCs and even beyond the range as extended by the present invention (through analysis of the colours revealed as the TLCs transition from transparent to colour and/or from colour to transparent). The spatial points or pixels making up the images may also be used to generate a curve fitting formula that allows interpolation of the subject or object temperature for any time points where no images were captured.

The temperature monitored or derived by the methods of the invention may be of an object or subject in contact with the TLC material, which method comprises analysing at least one heatmap generated from contacting the object or subject with the TLC material and monitoring or deriving the temperature of the object or subject from analysis of any one or more colours revealed as the TLCs transition from transparent to colour and/or from colour to transparent.

The TLCs may suitably be applied to the surface of a substrate, for example by spraying, painting, or printing, and/or the TLCs may be embedded within a substrate. The substrate may be any inert substrate, such as glass, plastic, resin, rubber etc. The TLCs may be comprised on or in at least a part of the substrate. The TLCs on or in the substrate may take the form of an array, such as dots or other shapes presented in an organised fashion. The substrate may indicate where contact with the subject or object should be made. For example, if the temperature to be detected is of a hand or foot, the substrate may guide the user on where to place their hand or foot.

According to a preferred embodiment, the TLCs are comprised in a thermochromic liquid crystal sheet (TLCS), for example, a commercially available TLCS.

The heatmap may be generated through the direct or indirect contact with the object or subject and the TLC material. Examples of indirect contact include where the TLCs are embedded in a substrate or where the subject is wearing socks, hosiery, gloves, a bandage or plaster.

The subject may be a mammal, optionally a human.

The TLC material may be comprised in a medical device. The medical device may be any known medical device incorporating TLCs for the purpose of monitoring and measuring temperature. In one embodiment, the medical device is a hand or foot monitor comprising TLC material, optionally arranged to allow generation of a heatmap. The TLC material may be a TLCS (thermochromic liquid crystal sheet), which TLCS may be commercially available or custom made. A heatmap may be generated by contacting the subject’s soles or palms with the TLC material. Contact with the TLCs may be direct or indirect, optionally the subject’s soles or palms are bare.

The term “heatmap” as defined herein refers to a display of at least two different colours on the TLC material, such as a substrate to which the TLCs are applied or embedded. Any reference herein to a “heatmap” may be to the heatmap when temporarily visible on the TLC material itself or to one or more images taken of the TLC material when the heatmap is visible, typically when the subject or object is still in contact with the TLC material or almost immediately following removal of contact.

The TLCs will be transparent below the starting point of the design range of the TLCs, revealing the colour of the substrate, which is often black. The TLCs will respond to a change in temperature by changing their specific arrangement (state, spacing etc.), which in turn will affect the wavelengths of light that can be absorbed and reflected off the crystals. Local differences in temperature correspond to local changes in colour, thus giving rise to a heatmap.

When a subject or object comes into contact with TLCs, the TLCs appearance changes as follows. At the lower end of their design temperature range TLCs are usually transparent (the smectic phase), revealing the colour of the substrate. Upon warming, the liquid crystals gradually lose transparency shifting into the chiral/cholesteric phase, blocking out the substrate colour and assuming a colour of their own. In a predominantly used formulation (the RGB type), this initial colour is a dark red that, with further warming, gradually shifts through the colours of a rainbow via orange, yellow, green and blue. At the upper end of the TLC design temperature range, the ultimate violet colour will then start to become increasingly transparent as the crystals shift into the isotropic phase, again revealing the substrate colour. Conventional methods and devices make use of the design temperature range defined by the manufacturer, i.e. the non-transparent sections between red and the beginning of violet (approximately between 0° and 290° in the HSV colour space). Conventional methods and devices do not make use of the transparent sections, i.e. between approximately 290° and 240° in the HSV colour space. The transition from colour to transparent (and vice versa) is not sudden but occurs gradually until full transparency is reached. Generally speaking, the present invention analyses the colours revealed during the transition from transparent to colour and/or from colour back to transparent.

The heatmap generated from contact of the subject or object is then analysed to determine the colours present. The analysis of the heatmap is ideally carried out on substantially the entire range of TLC colours and includes the colours revealed during the transition from (completely) transparent to (full) colour and/or from (full) colour to (completely) transparent. Alternatively, only a section or sections of the heatmap may be analysed. This could, for example, be an area indicating a hot spot or cold spot or any other area of particular interest. The analysis may exclude colours starting from the transparent to red transition to the yellow to green hues, which hues may represent a range from between about 0° to 90° in the HSV colour space, where 0° is pure red. Partial or total exclusion of these hues from the analysis suitably reduces the influence of spurious and unwanted artefacts, which may arise from reflections of light sources or from any measurement device.

The temperature monitoring or measurement of the invention may be performed on any known device incorporating TLCs or a TLCS for monitoring temperature.

A second aspect of the invention provides a method for monitoring or measuring hand and foot temperature in a subject, comprising (i) contacting one or both hands and/or one foot or both feet of a subject with TLC material, wherein said contacting generates a heatmap; (ii) analysing the heatmap for colours revealed as the TLCs transition from transparent to colour and/or from colour to transparent, including analysis of colours imperceivable by the human eye and/or colours extending beyond the temperature range set by the TLC manufacturer; (iii) monitoring or measuring points of elevated temperature (“hotspots”) and/or reduced temperature (“cold spots”) on said heatmap.

The TLC material may comprise a TLCS or TLCs comprised on or in a substrate.

The subject may be a mammal, preferably a human.

Preferably, the subject makes contact with the TLC material, which may be a TLCS or TLCs comprised on or in a substrate, with his bare hands or feet. Although the method of the invention would work through socks or hosiery, bare hands and feet are preferred for increased accuracy.

The presence of hotspots or colds spots may be determined by analysing the heatmap generated for anomalies, by comparing the heatmap generated to itself or to one or more previous heatmaps taken from the same subject of the same body part, or by comparing with heatmaps from healthy individuals, or by comparing to standard or healthy temperature values.

The presence of a hotspot may be an early warning sign of an emerging ulcer. The presence of a cold spot may be indicative of poor circulation and possible deterioration of an underlying condition such as Raynaud’s Phenomenon.

If a hotspot or cold spot is identified from the heatmap, the subject may seek guidance from a healthcare professional, take suitable medication, apply a suitable topical formulation, cold or warm compress, elevate the foot or feet, take rest. The device itself may be configured to advise the subject on the next course of action and/or to alert the healthcare professional to advise on the next course of action.

The temperature monitoring and derivation from the heatmap(s) or TLC materials may be computer-implemented for speed, increased accuracy and efficiency. The aforementioned alert systems may also be computer-implemented.

The present invention may also provide data processing means for carrying out the methods of the invention.

The present invention also provides a computer program comprising instructions, which when the program is executed by a computer, causes the computer to carry out any of the methods of the invention.

Conventional TLCS typically comprise four layers bonded together to form a sheet. The first layer is a thin sheet of transparent plastic material. This sheet is coated with an emulsion of liquid crystals; this forms the second layer. Once the emulsion has dried, the resulting liquid crystal layer is coated with black paint (layer three). The purpose of this third layer is to provide a strongly contrasting background for the colours produced by the liquid crystals. The fourth layer is typically a backing sheet which is applied to the black paint once dried. This backing sheet may be transparent or opaque as its only purpose is to protect the inner two layers from physical and chemical damage. In contrast, the first sheet must be transparent to allow the colours of the liquid crystals to be viewed through it. In some cases, the black paint layer (layer three) is omitted and in such cases the backing sheet is black. In some cases, there may be several layers of liquid crystal emulsions with different temperature characteristics applied one on top of the other to allow the temperature range to be extended.

When measuring the temperature of a hand or a foot using a conventional TLCS, the outline and area of the hand or foot is difficult to accurately decipher, particularly when the TLCS is transparent, i.e. when the temperature of the object in contact with the sheet falls outside of the measurement range for the TLCS. Similarly, the outline and area of any parts of the hand or foot that are of the same temperature as the TLCS will be difficult to determine; such parts will show as being the same colour as the TLCS.

With conventional TLCS being opaque, it is often not possible or at least difficult to generate an accurate representation of the hand or foot (or other contact-making object) and in turn difficult to derive accurate temperature values.

When using conventional TLCS, another difficulty is in ensuring that the TLCS image (of the hand or foot in this example) is overlayed or superimposed in the correct position onto the real ‘photo’ image of the corresponding hand or foot. Without accurate positioning, errors can occur and lead to an inaccurate determination of the temperature of the different parts of the hand or foot.

The present invention aims to address these issues through the use of electrochromic material.

A third aspect of the invention therefore provides a thermochromic liquid crystal sheet (TLCS), comprising: (a) TLC material applied to the surface of a substrate and/or embedded in a substrate, and (b) a layer of electrochromic material.

The electrochromic material may replace the black (contrast) paint layer and, if used, the backing sheet. Electrochromic material is well known in the art. Examples include Polymer Dispersed Liquid Crystal (PDLC) films, sometimes applied as a film to car windows (https://www.homewellcn.com/product/smart-film-for-car-12/). Manufacturers include Anhui Noyark Industry Co., Ltd, China (https://www.ahnoyark.com/intelligens-folia-pdlc-tint-film-black.html).

The substate to which the TLC material is applied is transparent to allow the colours of the liquid crystals to be viewed through it as well as to enable real photo images of the object or subject to be taken.

The electrochromic material may take the form of a (flexible) sheet. The electrochromic material may be laminated to or otherwise applied to the substate to which the TLC material is applied.

Use of the TLCS according to the third aspect of the invention allows the outline and area of the hand or foot (or other contact-making object) to be accurately determined. This makes use of the property of electrochromic materials to exist in an opaque state or a transparent state, effected by applying a current through the material for opacity or by removing the current for a transparent state. In use, when the TLCS according to the third aspect of the invention is transparent, in part or whole, the electrochromic material is switched to its transparent state. This allows the hand or foot (or other contact-making object) to be imaged through the now completely transparent TLCS structure, providing an exact image and outline of the hand or foot.

When the electrochromic material layer is switched back to its opaque state, it provides the necessary dark, contrasting background required to see and to image the colours of the liquid crystal layer on a heatmap. Areas showing without colour on the heatmap can either be due to there being no object present, for example, amputated toes, or may be due to the toes simply being of the same temperature as the TLCS or being of a temperature falling outside of the range of the liquid crystal layer. A comparison of the heatmap and the ‘real’ image taken when the electrochromic material was in transparent mode, allows it to be determined if the transparent regions even belong to an object (for example, part of a hand or foot).

If the transparent regions do belong to an object (for example, part of a hand or foot), such objects or parts would either be of the same temperature as the TLCS or would be of a temperature outside the design range of the liquid crystal material. Such parts can then be outlined and separated from any areas where the object is not in contact with the TLCS (for example, the arch of a foot), again by switching the electrochromic material to transparent.

When a contact-making object, say, a foot is placed on a TLCS according to the third aspect of the invention and the electrochromic material is in transparent mode, and where the temperature of the foot (or parts of it) are within the design temperature range of the TLCs, there will be a faint hue from the TLCs (faint due to the lack of a contrasting background). This faint hue can be corrected by image processing, for example, by switching the electrochromic material to opaque, recording the colours and switching it back to transparent and then deducting the recorded colours.

Advantageously, the TLCS according to the third aspect of the invention allows the contact-making object (for example a hand or foot) to be visually inspected through the TLCS when the electrochromic material is in transparent mode.

Another advantage of the TLCS according to the third aspect of the invention, is that the real photo image and thermal heatmap images of the object are in perfect spatial registration. This allows the unambiguous one-to-one association of parts of the real image with corresponding parts of the heatmap, thereby allowing for accurate temperature determination.

A TLCS according to the third aspect of the invention may be used in the methods according to the first and second aspects of the invention.

A fourth aspect of the invention provides the use of TLC material for monitoring or deriving the temperature of an object or subject in contact therewith, wherein the use comprises analysing any one or more colours revealed as the TLCs transition from transparent to colour and/or from colour to transparent from the contact and wherein the analysis includes analysis of colours imperceivable by the human eye and/or colours extending beyond the temperature range set by the TLC manufacturer.

The invention also provides use of a TLCS according to the third aspect of the invention, wherein said use allows for an accurate outline and area of the hand or foot (or other contact-making object) to be accurately determined and/or allows for visual inspection of the contact-making object when the electrochromic material is in transparent mode.

TLCs are used for temperature monitoring and measurement in a wide range of areas. For example, TLCs may be found in thermometers for use in refrigerators, aquariums, forehead thermometers, infant baths and bottles. TLCs are also often used in manufacturing, for example, in process control, machine monitoring etc. TLCs may also be found in a variety of medical devices. In one example, the TLCs are comprised in a device for monitoring hand or foot health. This is especially important for the diabetic population and for other individuals with conditions such as Raynaud’s phenomenon. There are known devices for monitoring foot health that incorporate the use of TLCs and TLCS. The advantage of such devices is that they can be used at home, between appointments and without the need for a healthcare professional. The devices could therefore potentially catch any problems early, avoiding costly hospital visits and progression of the condition to a potentially life-changing or irreversible state. Such devices will only be used by the patient population or recommended by healthcare workers if they are sufficiently accurate and at the right price point and easy to use. The use of off-the-shelf TLCS will reduce the cost of such devices, but off-the shelf TLCS have the drawbacks relating to measurable range and levels of accuracy discussed above. The methods and use of the invention advantageously allows more accurate temperature information to be derived from conventional TLCs and off-the-shelf TLCS due to the reduction in artefacts and due to extension of the usable temperature range set by manufacturers. The use of these methods would enhance known medical devices, such as foot monitors, making the devices more useful.

The use according to the fourth aspect of the invention may take place on TLCs or TLCS comprised in manufacturing machinery or in a medical device.

A fifth aspect of the present invention provides a system for monitoring or measuring the temperature of a subject or object, comprising:
(i) TLC material, wherein contacting of the subject or object with the TLC material generates a heatmap; and
(ii) optionally a camera for capturing at least one image of the heatmap, preferably wherein multiple images are captured, such as through a continuous image acquisition process; and
(iii) computer means for analysing colours revealed on the heatmap as the TLCs transition from transparent to colour and/or from colour to transparent, including analysis of colours imperceivable by the human eye and/or colours extending beyond the temperature range set by the TLC manufacturer, and monitoring or deriving the temperature of the object or subject by correlating the colours revealed to a temperature value.

The system may further comprise illumination means. Such illumination means may help reduce shadows and improve the quality of the image.

The TLC material may comprise a TLCS or TLCs comprised on or in a substrate. The TLCS may be as described in the third aspect of the invention. The camera in that case may additionally capture at least one real image of the subject or object.

The analysis may exclude colours starting from the transparent to red transition to the yellow to green hues, which hues may be represented by a range from between about 0° to 90° in the HSV colour space, where 0° is pure red. Partial or total exclusion of these hues from the analysis suitably reduces the influence of spurious and unwanted artefacts, which may arise from reflections of light sources or from any measurement device.

When using a TLCS according to the third aspect of the invention, the computer means may additionally correct for hue and/or monitor and record an outline of the contact-making object or parts thereof, as described hereinabove.

The computer means may further monitor for points of elevated temperature (“hotspots”) and/or reduced temperature (“cold spots”) on said heatmap.

The system may be configured to alert the subject to perform an action if a hotspot or cold spot is identified from the heatmap. For example, the subject may be alerted to seek guidance from a healthcare professional, to take suitable medication, apply a suitable topical formulation or to elevate the leg or to take rest. The system may be configured to advise the subject on the next course of action and/or to alert the healthcare professional to advise on the next course of action.

### BRIEF DESCRIPTION OF THE DRAWINGS

One or more embodiments of the invention will now be described, by way of example only, with reference to the accompanying drawings, in which:

FIG. 1 is a schematic representation of a device for measuring temperature accordance with the invention.

FIG. 2 illustrates the colour transitions of a typical TLCS.

FIG. 3 is a flow chart of steps carried out in one embodiment by the device of the invention.

FIG. 4 shows how artefacts are reduced by excluding TLCS colours associated with lower temperatures.

FIG. 5 is a flow chart detailing the image processing steps carried out in one embodiment to derive temperature values from the images.

FIG. 6 shows a temperature extrapolation method used to estimate temperatures outside the TLCS temperature range.

FIG. 7 is a representation of a TLCS comprising a sheet of electrochromic material.

The present invention will now be described with reference to the following non-limiting examples. Furthermore, elements or components that are described with reference to any Figure may be interchanged with those of other Figures or other equivalent elements without departing from the spirit of the present teaching.

FIG. 1 shows a schematic representation of a temperature measuring device 100 for measuring the temperature of subject or object 105. The device 100 comprises an enclosure 101 with transparent panel 102 on which a TLCS 103 is placed. The TLCS has one surface comprising the thermochromic liquid crystal layer, which surface faces camera 104, and another surface comprising backing material which comes into direct contact with subject or object 105. A computer 106 is operatively connected to camera 104 and one or more optional light sources 107 for illuminating the TLCS. An optional proximity sensor 108 informs the computer 106 when a subject or object 105 makes contact with the TLCS 103.

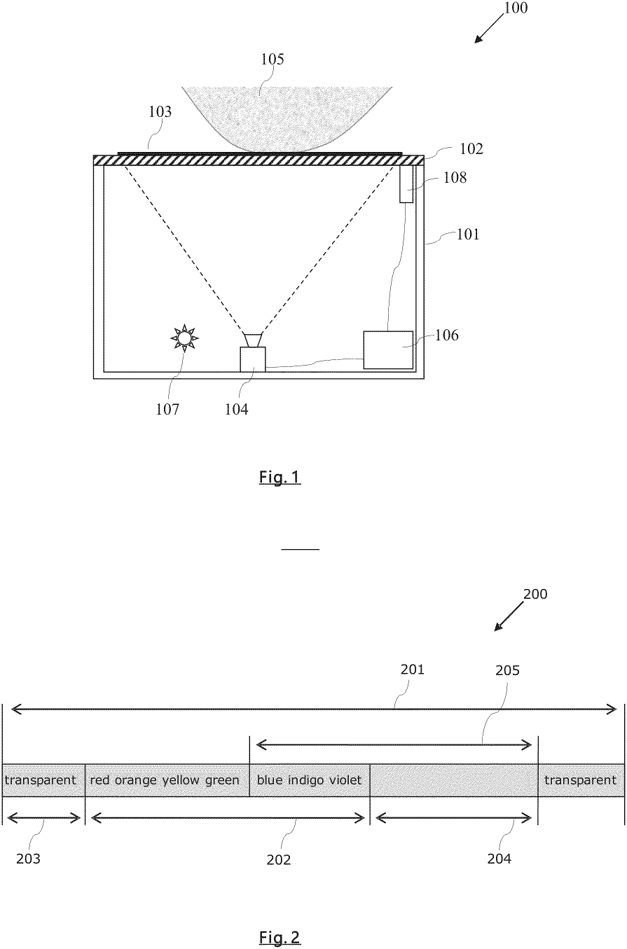

FIG. 2 shows the colour gamut 200 produced by TLCS 103. The full gamut 201 includes all the colours of the rainbow or the visible spectrum 202, which are discernible by the naked eye and constitute the temperature range for off-the shelf TLCS. The outermost rainbow colours red (at the lower end of the spectrum) and violet (at the higher end of the spectrum) eventually fade into transparency, revealing the colour of the TLCS substrate backing material. The temperature interval is short for the transparent to red transition 203. For the violet to transparent transition 204 the interval is significantly longer, in some TLC materials it can be longer than the design interval 202 set by manufacturers. The range 205 indicates an example of the subrange of colours analysed in the methods of the invention.

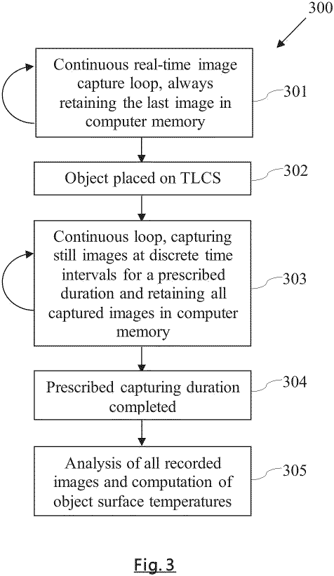

FIG. 3 shows an example flowchart 300 of steps when using the device 100. In 301 the computer 106 receives a continuous real-time image stream from camera 104 and retains the respective last image of the stream in the computer’s memory. In 302 sensor 108 signals the presence of object or subject 105 on the TLCS surface 102 and the continuous loop terminates. In 303 the computer 106 immediately enters into a second continuous loop where it captures and stores in computer memory still images from camera 104 at discrete time intervals. In 304, when a prescribed amount of time has passed, the second continuous loop is terminated. In 305, the computer immediately commences with the analysis of the images stored in the memory, including the image stored previously in 301.

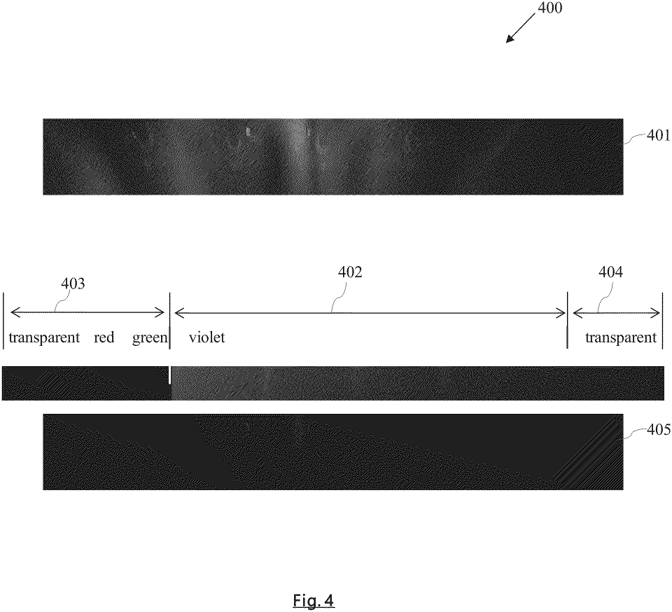

FIG. 4 shows the use of a subsection of the full colour gamut 201. 401 is an enlarged view of TLCS 103 in device 100 captured by camera 104. The TLCS sheet is not in contact with subject or object 105 and is therefore only exposed to ambient temperatures surrounding device 100. A temperature which is below the point where the TLCS colour transition from transparent to red occurs. 401 should therefore show only the substrate colour of TLCS 103, which is black in this instance. However, 401 shows spurious features such as highlights and reflections emanating in this case from the illumination 107 inside device 100.

Restricting image processing by the computer 106 to the colour gamut shown in 402, excluding regions 403 and 406, results in a processed version of the previous area 405 where the amount spurious features is significantly reduced.

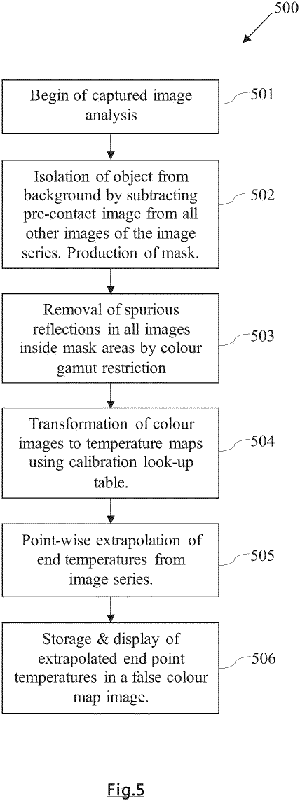

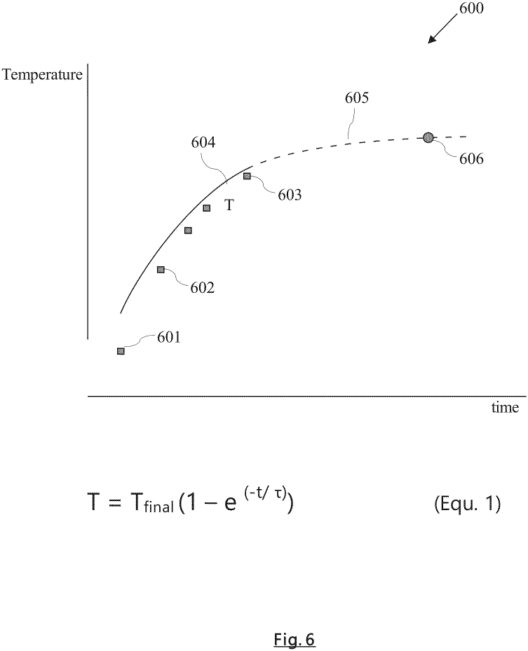

FIG. 5, flowchart 500, shows the steps involved in processing the images stored in the computer memory as described above and shown in 301 and 303 of FIG. 3. Any time after completion of block 305 the processing starts: block 501 of FIG. 6.

In a first step, the pre-contact image stored in block 301 is pixel-wise subtracted from all images stored in block 303. This isolates the object in these images. Standard image processing algorithms known to persons having ordinary skill in the art are then used to create a mask from the isolated object. Further processing of all images is restricted to the area covered by the mask, block 502.

By using only a subsection of the full colour gamut 201 spurious features are removed from all images, block 503.

A calibration look-up table of the sort known to persons having ordinary skill in the art is then used to pixel-wise translate colour values into a temperature map for each image, block 504.

Using the known time interval between image captures in continuous loop 303 a pixel-wise extrapolation of the values in the temperature maps resulting from block 504 can be performed by applying measurement task-appropriate curve-fitting algorithms. Such extrapolation can produce pixel-wise approximations of the eventual temperatures at points that lie beyond the upper limit of the extended TLCS temperature range 205, block 505.

The extrapolated temperature values generated in block 505 are stored in computer memory for further use such as permanent storage onto computer readable media. They may also be displayed in a variety of formats known to persons having ordinary skill in the art, including in the form of a false-colour image, block 506.

The graph 600 of FIG. 6 illustrates an example for estimating temperatures outside both the designed temperature range of TLCS 202 and the extended range 204. Measurement point 601 results from the retained last image in computer memory in block 301. Several more points between 602 and 603 result from the continuous loop in block 303.

Using knowledge of the underlying mechanism of the heat transfer between object 105 into the TLCS 103 and the transparent panel 102 an approximating equation can be derived that results in curve T, 604. In most cases this curve will be an inverse exponential function of the generalised form provided in equation (1) but other functions are possible, e.g. in the case of actively heated or cooled objects 105.

The equation (1) describing curve T 604 can then be used to interpolate the temperature measured for object 105 for any moment in between the discrete measurement points 601 to 603 and to extrapolate the temperature for any later point in time such as 606.

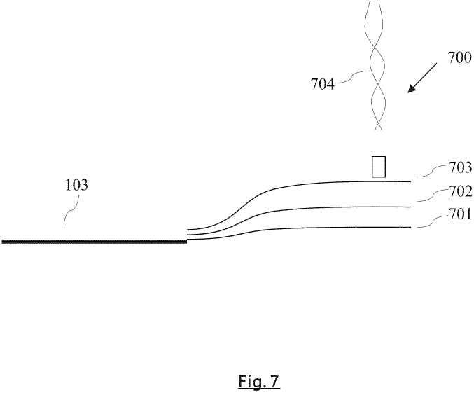

FIG. 7 shows a TLCS 103 comprising a transparent bottom sheet 701 in contact with transparent panel 102. One or more layers of liquid crystal material 702 forms the next layer. A sheet of electrochromic material 703 forms the topmost layer. Together with the bottom sheet 701 it protects the liquid crystal material from chemical or mechanical damage. The electrochromic sheet changes it optical property from opaque to transparent by applying a control voltage via electrical contacts 704. All three layer of TLCS 103 are bonded together either by an adhesive or, in the case of the liquid crystal layer 702 by adhesion of the liquid crystal paint to its substrate.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

1-30. (canceled)

31. A method for monitoring or measuring temperature using thermochromic liquid crystal (TLC) material, comprising analysing colours revealed as the TLCs transition from transparent to colour and/or from colour to transparent, including colours imperceivable by the human eye and/or colours extending beyond the temperature range set by a TLC manufacturer.

32. The method according to claim 31, wherein said analysis of colours extends a lower and/or an upper end of the nominal temperature range of the TLC material.

33. The method according to claim 32, wherein the temperature range is extended beyond the lower end of the nominal range of the TLC material by analysis of the colours revealed as the TLCs transition from transparent to red.

34. The method according to claim 33, wherein the colours include hues represented by approximately 330° to 355° in a Hue Saturation Value (HSV) colour space, where 0° is pure red.

35. The method according to claim 32, wherein the temperature range is extended beyond the upper end of the nominal range of the TLC material by analysis of the colours revealed as the TLCs transition from violet to transparent.

36. The method according to claim 35, wherein the colours include hues represented by approximately 240° to 360°, preferably 290° to 355°, in the Hue Saturation Value (HSV) colour space, where 0° is pure red.

37. The method according to claim 31, wherein the colour analysis at least partially excludes colours starting from the transparent to red transition to yellow to green hues.

38. The method according to claim 37, wherein which hues are represented by a range from between about 0° to 90° in the HSV colour space, where 0° is pure red, and wherein said exclusion reduces artefacts.

39. The method according to claim 31, wherein said colour analysis comprises analysing only a Hue (H) component of a Hue/Saturation/Value (HIV) colour space and determining temperature from said H component alone.

40. The method according to claim 31, wherein said analysis is carried out on at least one image of said TLC material.

41. The method according to claim 31, wherein specific colours of the TLCs are correlated to specific temperatures by reference to a calibration table or curve fitting formula.

42. The method according to claim 41, wherein said curve fitting formula allows an extrapolation and/or an interpolation of temperature values.

43. The method according to claim 31, wherein the temperature monitored or measured is of an object or subject in contact with TLC material, said method comprising analysing at least one heatmap generated by contacting said object or subject with said TLC material and monitoring or deriving the temperature of the object or subject from analysis of any one or more colours revealed as the TLCs transition from transparent to colour and/or from colour to transparent, wherein said analysis includes colours imperceivable by the human eye and/or colours extending beyond the temperature range set by the TLC manufacturer, wherein said contact is direct or indirect, wherein the subject is a mammal.

44. A method for monitoring or measuring hand or foot temperature in a subject, comprising (i) contacting hand(s) or foot/feet of a subject with a substrate comprising thermochromic liquid crystals (TLCs), wherein said contacting generates a heatmap on said substrate; (ii) analysing the heatmap for colours revealed as the TLCs transition from transparent to colour and/or from colour to transparent, wherein said analysis includes colours imperceivable by the human eye and/or colours extending beyond the temperature range set by the TLC manufacturer; and (iii) monitoring for points of elevated temperature (“hotspots”) and/or reduced temperature (“cold spots”) on said heatmap.

45. The method according to claim 44, wherein a presence of hotspots or colds spots is determined by analysing the heatmap generated for anomalies by comparing the heatmap generated to itself or to one or more previous heatmaps taken from the same subject and/or to reference heatmaps or temperature values.

46. The method according to claim 44, wherein said TLC material is applied to a surface of a substrate and/or is embedded in a substrate.

47. The method according to claim 46, wherein the TLC material is comprised in a thermochromic liquid crystal sheet (TLCS).

48. The method according to claim 46, wherein said TLCS additionally comprise a black layer or another colour suitable for providing contrast.

49. The method according to claim 48, wherein said black or contrasting layer is replaced by a layer of electrochromic material.

50. A thermochromic liquid crystal sheet (TLCS), comprising (a) TLC material applied to the surface of a substrate and/or embedded in a substrate, and (b) a layer of electrochromic material.
